(19) United States
(12) Patent Application Publication
(10) Pub. No.: US 2021/0244287 A1
(43) Pub. Date: Aug. 12, 2021

(54) HEARTBEAT DETECTION DEVICE, HEARTBEAT DETECTION METHOD, AND PROGRAM

(71) Applicant: MURAKAMI CORPORATION, Shizuoka (JP)

(72) Inventor: Atsushi HAYAMI, Shizuoka (JP)

(73) Assignee: MURAKAMI CORPORATION, Shizuoka (JP)

(21) Appl. No.: 16/973,634

(22) PCT Filed: Jun. 3, 2019
(86) PCT No.: PCT/JP2019/021961

§ 371 Date: Dec. 9, 2020

(30) Foreign Application Priority Data
Jun. 28, 2018 (JP) 2018-122754

(51) Int. Cl. A61B 5/024; A61B 5/00

(52) U.S. Cl. CPC A61B 5/024; A61B 5/7235; A61B 5/0059

(57) ABSTRACT

The accuracy of detection of a heartbeat is increased and a time for detection of a heartbeat is shortened. A heartbeat detection device includes a heartbeat detection unit which detects a heart rate using the luminance of captured images of a part of a body surface of a user, which are captured images of a plurality of frames which have been captured in chronological order. The heartbeat detection unit computes a total luminance of the captured image of each of the frames, delays a vibrating wave representing chronological change of the total luminance at certain time intervals, and computes the heart rate using a cycle of a peak at which a difference between the vibrating wave which has not been subjected to a delay and each vibrating wave which has been subjected to a delay is reduced in a waveform of the difference.

### TECHNICAL FIELD

The present invention relates to a heartbeat detection device, a heartbeat detection method, and a program.

### BACKGROUND ART

In the related art, evaluating stress by detecting a heart rate from a captured image of a user has been performed. Since a heart rate can be measured without bringing a device into contact with a body surface of a user, it is possible to easily evaluate stress.

As a detection method for a heart rate, for example, a method for detecting a pulse by obtaining heartbeat interval data from temporal change of a pixel average value of a captured image from which pigment components have been separated and performing frequency conversion on the obtained heartbeat interval data has been proposed (for example, refer to Patent Literature 1).

### CITATION LIST

### Patent Literature

[Patent Literature 1]

Japanese Patent Laid-Open No. 2017-29318

### SUMMARY OF INVENTION

### Technical Problem

However, the luminance of a captured image changes significantly even if a user moves a little. Since frequency conversion is easily affected by a long-cycle component such as the movement of a user, the accuracy of detection of a heart rate easily deteriorates. In order to obtain sufficient accuracy of detection, it is necessary to increase the number of frames of the captured image, which increases an amount of data and an amount of computation and prolongs a time for detection of a heart rate.

An object of the present invention is to increase the accuracy of detection of a heart rate and shorten a time for detection of a heart rate.

### Solution to Problem

According to the invention disclosed in claim 1, there is provided a heartbeat detection device including:

a heartbeat detection unit which detects a heart rate using the luminance of captured images of a part of a body surface of a user, which are captured images of a plurality of frames which have been captured in chronological order,

wherein the heartbeat detection unit computes a total luminance of the captured image of each of the frames, delays a vibrating wave representing chronological change of the total luminance at certain time intervals, and computes the heart rate using a cycle of a peak at which a difference between the vibrating wave which has not been subjected to a delay and each vibrating wave which has been subjected to a delay is reduced in a waveform of the difference.

According to the above-described heartbeat detection device, since a vibrating wave component having the periodicity of a heartbeat is obtained from the difference between the vibrating wave which has not been subjected to a delay and each vibrating wave which has been subjected to a delay, even when a vibrating wave component of a long cycle due to the movement of the user is included in the vibrating wave, it is possible to detect a heart rate with high accuracy. Furthermore, since a heart rate can be computed through simple computation of the addition of the luminance and the subtraction of each vibrating wave, it is possible to detect a heart rate with a small amount of computation. Therefore, it is also possible to shorten a time for detection of a heart rate.

According to the invention disclosed in claim 2, there is provided the heartbeat detection device according to claim 1,

wherein the heartbeat detection unit computes the heart rate using a period from a time at which the waveform of the difference starts to a time at which the peak appears first, as one cycle.

Thus, it is possible to reduce an influence of vibrating waves other than a heartbeat to obtain a cycle of a heartbeat and the accuracy of detection of a heart rate is further improved.

According to the invention disclosed in claim 3, there is provided the heartbeat detection device according to claim 1 or 2 further including:

a determination unit which determines a reliability of the heart rate detected by the heartbeat detection unit and outputs the reliability together with the heart rate.

Thus, it is possible to provide a heart rate as well as the reliability of the heart rate.

According to the invention disclosed in claim 4, there is provided the heartbeat detection device according to any one of claims 1 to 3,

wherein the luminance is a luminance in green.

Thus, the sensitivity to hemoglobin, whose amount changes in accordance with a pulsebeat, is improved and the accuracy of detection of a heart rate is further improved.

According to the invention disclosed in claim 5, there is provided the heartbeat detection device according to any one of claims 1 to 4 further including:

a region of interest (ROI) setting unit which sets an ROI in the captured image,

wherein the heartbeat detection unit computes a total luminance in the ROI.

Thus, it is possible to reduce an amount of computation of the total luminance and further shorten a time for detection of a heart rate.

According to the invention disclosed in claim 6, there is provided the heartbeat detection device according to any one of claims 1 to 5,

wherein the captured image is a captured image of a face of the user, and

the heartbeat detection device includes:

a feature point extraction unit which extracts a feature point of the face in the captured image of each of the frames; and

a tracking unit which uses the feature point to adjust a face position in the captured image of each of the frames.

Thus, it is possible to reduce a noise component due to the movement of the user and the accuracy of detection of a heart rate is further improved.

According to the invention disclosed in claim 7, there is provided a heartbeat detection method including:

detecting a heart rate using the luminance of captured images of a part of a body surface of a user, which are captured images of a plurality of frames which have been captured in chronological order,

wherein the detecting the heart rate includes:

computing a total luminance of the captured image of each of the frames;

delaying a vibrating wave representing chronological change of the total luminance at certain time intervals; and

computing the heart rate using a cycle of a peak at which a difference between the vibrating wave which has not been subjected to a delay and each vibrating wave which has been subjected to a delay is reduced in a waveform of the difference.

According to the above-described heartbeat detection method, since a vibrating wave component having the periodicity of a heartbeat is obtained from the difference between the vibrating wave which has not been subjected to a delay and each vibrating wave which has been subjected to a delay, even when a vibrating wave component of a long cycle due to the movement of the user is included in the vibrating wave, it is possible to detect a heart rate with high accuracy. Furthermore, since a heart rate can be computed through simple computation of the addition of the luminance and the subtraction of each vibrating wave, it is possible to detect a heart rate with a small amount of computation. Therefore, it is also possible to shorten a time for detection of a heart rate.

According to the invention disclosed in claim 8, there is provided a program causing a computer to execute:

detecting a heart rate using the luminance of captured images of a part of a body surface of a user, which are captured images of a plurality of frames which have been captured in chronological order,

wherein the detecting the heart rate includes:

computing a total luminance of the captured image of each of the frames;

delaying a vibrating wave representing chronological change of the total luminance at certain time intervals; and

computing the heart rate using a cycle of a peak at which a difference between the vibrating wave which has not been subjected to a delay and each vibrating wave which has been subjected to a delay is reduced in a waveform of the difference.

According to the above-described program, since a vibrating wave component having the periodicity of a heartbeat is obtained from the difference between the vibrating wave which has not been subjected to a delay and each vibrating wave which has been subjected to a delay, even when a vibrating wave component of a long cycle due to the movement of the user is included in the vibrating wave, it is possible to detect a heart rate with high accuracy. Furthermore, since a heart rate can be computed through simple computation of the addition of the luminance and the subtraction of each vibrating wave, it is possible to detect a heart rate with a small amount of computation. Therefore, it is also possible to shorten a time for detection of a heart rate.

### Advantageous Effects of Invention

According to the present invention, it is possible to increase the accuracy of detection of a heart rate and shorten a time for detection of a heart rate.

### BRIEF DESCRIPTION OF DRAWINGS

FIG. 1 is a block diagram illustrating a configuration of a heartbeat detection device according to an embodiment of the present invention for each function.

FIG. 2 is a diagram illustrating an example of a feature amount extracted from a face image.

FIG. 3 is a graph for describing an example of a vibrating wave representing chronological change of a total luminance.

FIG. 4 is a graph for describing a vibrating wave on which correction has been performed.

FIG. 5A is a graph for describing an example of a vibrating wave which has not been subjected to a delay and each vibrating wave which has been subjected to a delay.

FIG. 5B is a graph for describing an example of a waveform of a difference between a vibrating wave which has not been subjected to a delay and each vibrating wave which has been subjected to a delay.

FIG. 6 is a graph for describing a waveform of a difference between a vibrating wave which has not been subjected to a delay and the vibrating wave which has been subjected to a delay.

FIG. 7 is a graph for describing a display example of a heart rate.

FIG. 8 is a flowchart for describing a processing procedure when a heartbeat detection device detects a heart rate.

### DESCRIPTION OF EMBODIMENTS

Hereinafter, embodiments of a heartbeat detection device, a heartbeat detection method, and a program of the present invention will be described below with reference to the drawings.

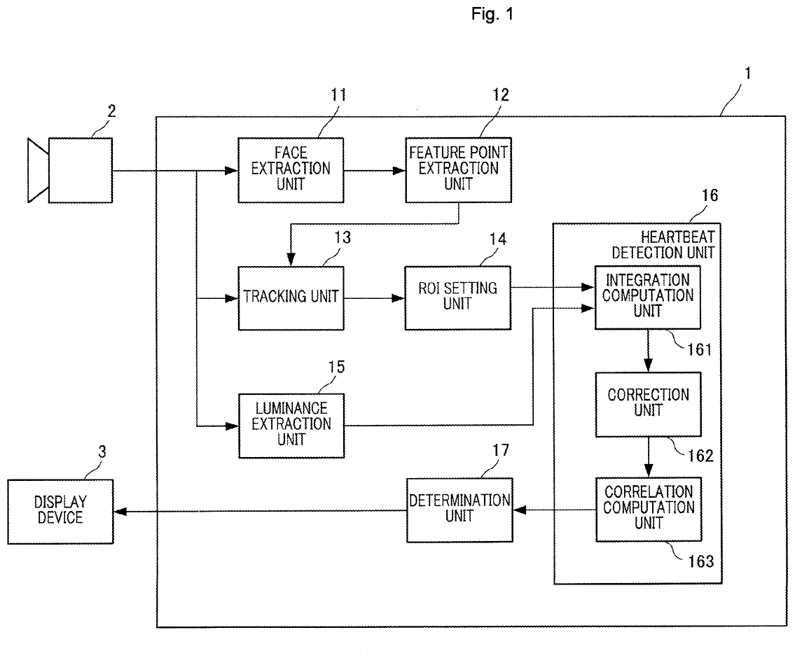

FIG. 1 is a block diagram illustrating a configuration of a heartbeat detection device 1 according to an embodiment of the present invention for each function.

As shown in FIG. 1, the heartbeat detection device 1 is connected to an imaging device 2 and detects a heart rate from captured images of a user input from the imaging device 2. Furthermore, the heartbeat detection device 1 is connected to a display device 3 and outputs the detected heart rate to the display device 3.

### Imaging Device

The imaging device 2 generates captured images of a part of a body surface of the user, which are captured images of a plurality of frames captured in chronological order. In this embodiment, the captured images are bitmap images in which each pixel has a luminance in R (red), G (green), and B (blue). Furthermore, the captured images are captured images of the user's face. If the face is included in the captured image, it is easy to adjust positions of the captured images among the frames on the basis of positions of facial feature points.

### Display Device

The display device 3 displays a heart rate output from the heartbeat detection device 1. As the display device 3, for example, a liquid crystal display (LCD), a touch panel, or the like can be used.

### Heartbeat Detection Device

As shown in FIG. 1, the heartbeat detection device 1 is configured to include a face extraction unit 11, a feature point extraction unit 12, a tracking unit 13, a region of interest (ROI) setting unit 14, a luminance extraction unit 15, a heartbeat detection unit 16, and a determination unit 17.

It is possible to implement the processing content of each constituent element unit of the heartbeat detection device 1 using hardware such as a field-programmable gate array (FPGA) and a large scale integration (LSI). Furthermore, it is possible to implement the processing content of each constituent element unit through software processing in which a computer reads a program having the processing procedure written therein from a storage medium having the program stored therein and executes the program. As the computer, for example, a processor such as a central processing unit (CPU) and a graphics processing unit (GPU) can be used. As the storage medium, a hard disk, a read only memory (ROM), or the like can be used.

The face extraction unit 11 extracts a face region of the user from a captured image input from the imaging device 2. A face recognition method using the face extraction unit 11 is not particularly limited and can be, for example, a known method such as template matching.

The feature point extraction unit 12 extracts a plurality of feature points from the face region extracted using the face extraction unit 11 and computes a feature amount of each of the feature points. A method for extracting a feature point which can be used is not particularly limited. In addition, examples thereof include corner feature amounts such as FAST and Harris, local feature amounts such as SURF and KAZE, gradient histograms, and the like.

The tracking unit 13 adjusts a face position of a captured image of each frame on the basis of a position of the feature point extracted by the feature point extraction unit 12. To be specific, the tracking unit 13 performs projective transformation on a captured image of a current frame so that positions of feature points having the highest degree of similarity in feature amount coincide with each other between the current frame and an immediately previous frame, which are input from the imaging device 2. Thus, it is possible to make a face position of the current frame conform to a face position of the immediately previous frame.

The ROI setting unit 14 sets an ROI in the captured image in which the tracking unit 13 made the face position adjusted. Although the ROI setting unit 14 can arbitrarily set a position and a size of the ROI, it is desirable to set a region including a region around a mouth or a region around a nose as an ROI. In the region around the mouth or around the nose, a change in amount of hemoglobin in the blood easily appears on the body surface, making it easy to detect a heart rate. It is possible to detect positions of the mouth and the nose in the captured image through template matching or the like.

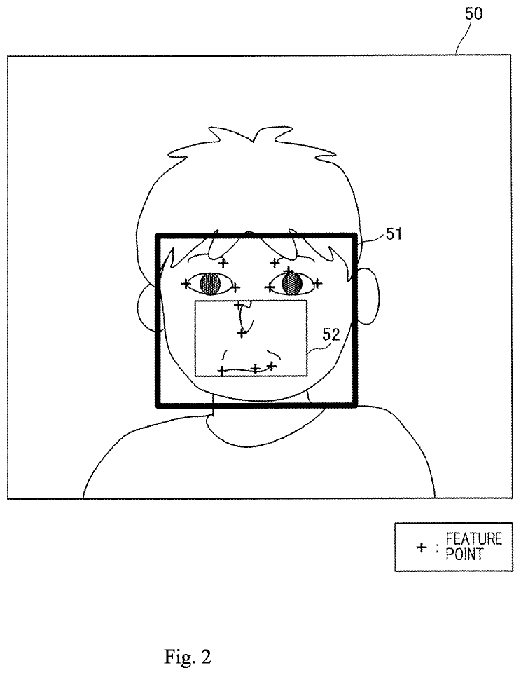

FIG. 2 illustrates an example of a captured image.

As shown in FIG. 2, a face region 51 is extracted from a captured image 50 and feature points are extracted. In FIG. 2, each of the feature points is represented by a cross-like marker. In the face region 51, a region 52 including a nose and a mouth is set as an ROI.

The luminance extraction unit 15 extracts the luminance to be used for detecting a heart rate of the luminance in R, G, and B of a captured image. Although a heartbeat can be detected also in the luminance in any color, it is desirable that the luminance extraction unit 15 extract the luminance in G. The luminance in G has a high sensitivity to hemoglobin, an amount of which changes in accordance with a pulsebeat and in this case the accuracy of detection of a heart rate is easily improved.

The heartbeat detection unit 16 computes a total luminance of a captured image of each of the frames and delays a vibrating wave representing chronological change of the total luminance at certain time intervals. The heartbeat detection unit 16 computes a heart rate from a difference between a vibrating wave which has not been subjected to a delay and each vibrating wave which has been subjected to a delay.

As shown in FIG. 1, the heartbeat detection unit 16 includes an integration computation unit 161, a correction unit 162, and a correlation computation unit 163.

The integration computation unit 161 computes a total luminance of a captured image of each of the frames. Although the integration computation unit 161 can also compute a total luminance of all regions of a captured image, it is desirable to compute a total luminance in an ROI set by the ROI setting unit 14. Thus, it is possible to reduce an amount of computation and shorten a time for detection of a heart rate.

When a total luminance computed from the captured image of each of the frames is plotted with respect to a capturing time of the captured image of each of the frames, a vibrating wave representing chronological change of the luminance is obtained. An amount of hemoglobin in the blood changes in accordance with a pulsebeat and the luminance of the captured image changes in accordance with the amount of hemoglobin. For this reason, the obtained vibrating wave includes a vibrating wave of a heartbeat.

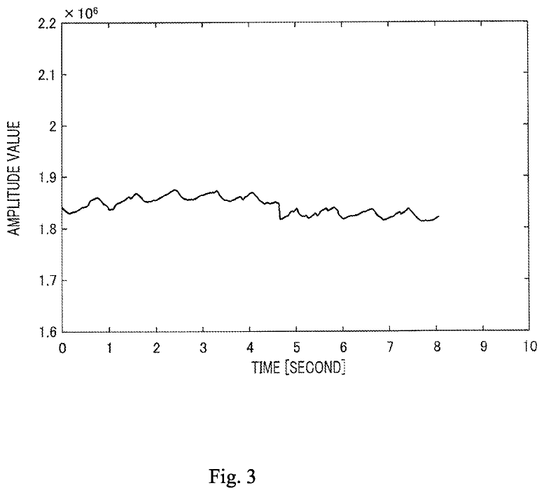

FIG. 3 illustrates an example of a vibrating wave representing chronological change of a total luminance of an ROI.

As shown in FIG. 3, a vibrating wave includes a periodic vibrating wave component.

The correction unit 162 corrects the vibrating wave obtained using the integration computation unit 161. The correction unit 162 performs filter processing on the vibrating wave as one correction and removes a vibrating wave component which does not affect a heartbeat. Although individual differences are present, generally, a frequency of a vibrating wave of a heartbeat is about 1 Hz and varies within the range of about 0.7 to 2.0 Hz in accordance with a physical condition. The correction unit 162 can remove a noise component which does not affect a heartbeat by extracting a vibrating wave component having a frequency near this range, for example, a vibrating wave component which is located in a frequency band of 0.1 to 2.8 Hz. Examples of filters which can be used for the filter processing include band pass filters, high pass filters, low pass filters, and the like.

Also, the correction unit 162 adjusts an amplitude of a vibrating wave to be constant by performing auto gain control (AGC) as one correction.

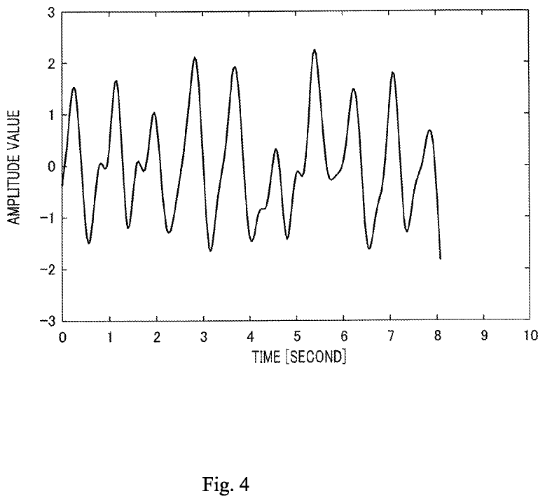

FIG. 4 illustrates a vibrating wave obtained by correcting the vibrating wave illustrated in FIG. 3.

As shown in FIG. 4, a vibrating wave which is a noise component is removed through the correction, and a vibrating wave in which a vibrating wave component of a heartbeat is highlighted is obtained.

The correlation computation unit 163 delays the vibrating wave obtained using the correction unit 162 at certain time intervals and computes a difference between a vibrating wave which has not been subjected to a delay and each vibrating wave which has been subjected to a delay. To be specific, the correlation computation unit 163 holds the vibrating wave obtained using the correction unit 162 in a memory such as a buffer memory and holds each of the vibrating waves delayed for a certain time in a memory such as a ring buffer memory. The correlation computation unit 163 computes a difference between the held vibrating wave which has not been subjected to a delay and each held vibrating wave which has been subjected to a delay.

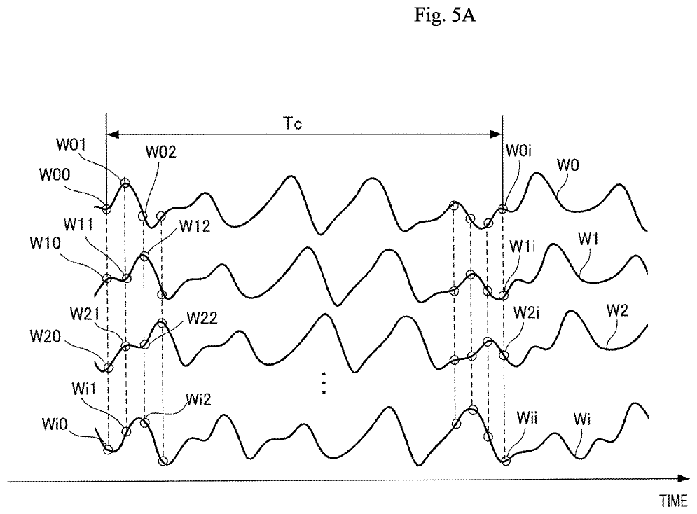

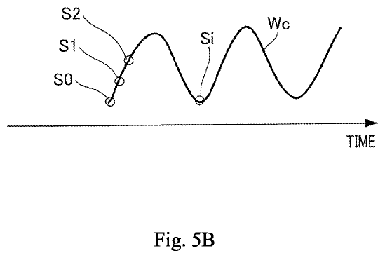

FIG. 5A illustrates an example of a vibrating wave which has not been subjected to a delay and each vibrating wave which has been subjected to a delay.

As shown in FIG. 5A, each vibrating wave Wi is obtained by delaying the original vibrating wave W0 by a time which is obtained by multiplying a certain time t by i (i is an integer of 1 or more). For example, the vibrating wave W1 is a vibrating wave obtained by delaying the vibrating wave WO by the certain time t and the vibrating wave W2 is a vibrating wave obtained by further delaying the vibrating wave W1 by the certain time t, that is, a vibrating wave obtained by delaying the vibrating wave W0 by a time 2t.

The correlation computation unit 163 compares the vibrating wave W0 which has not been subjected to a delay with each vibrating wave Wi which has been subjected to a delay within a computation period Tc and calculates a difference therebetween.

The computation period Tc can be determined in accordance with a cycle of a heartbeat to be detected. For example, when a heartbeat with a heart rate of 30 BPM or more is detected, one cycle is about 2 seconds. Thus, it is desirable to determine the computation period Tc to be 4 seconds or more, which is at least two cycles or more.

To be specific, the correlation computation unit 163 samples the vibrating wave W0 which has not been subjected to a delay and each vibrating wave Wi which has been subjected to a delay at constant sampling intervals within the computation period Tc. The sampling intervals are times which are the same as an amount of delay of each vibrating wave Wi. As will be represented by the following expression, the correlation computation unit 163 calculates a total Sj of absolute values of differences between a sampled vibrating wave W0j which has not been subjected to a delay and each sampled vibrating wave Wij which has been subjected to a delay. j represents the number of times of sampling and j=0 to i.

Sj=Σ{abs(W0j−Wij)}

In the foregoing expression, abs( ) represents a function in which an absolute value of the computation result in ( ) is output. W0j indicates an amplitude value of the sampled vibrating wave W0 which has not been subjected to a delay. Wij indicates an amplitude value of each sampled vibrating wave Wi which has been subjected to a delay.

FIG. 5B illustrates a waveform of a total Sj of an absolute value of a difference.

For example, S0, S1, S2, . . . , S1 in FIG. 5B are calculated from the vibrating waves W0 to Wi illustrated in FIG. 5A as follows:

$$S 0 = abs ( W 00 - W 00 ) + abs ( W 01 - W 01 ) + … + abs ( W 0 i - W 0 i ) ;$$

$$S 1 = abs ( W 00 - W 10 ) + abs ( W 01 - W 11 ) + … + abs ( W 0 i - W 1 i ) ;$$

$$S 2 = abs ( W 00 - W 20 ) + abs ( W 01 - W 21 ) + … + abs ( W 0 i - W 2 i ) ;$$

$$…$$

$$S i = abs ( W 00 - W i 0 ) + abs ( W 01 - Wi 1 ) + … + abs ( W 0 i - W ii ) .$$

When a vibrating wave, which is periodic such as a heartbeat, is delayed for a certain time, a difference from the original vibrating wave becomes large, but when the vibrating wave is further delayed and has a cycle coinciding with that of the vibrating wave itself, the difference becomes smaller. For this reason, as shown in FIG. 5B, when Sj is output at the same sampling interval as a delay time, it is possible to obtain the original vibrating wave W0, that is, the vibrating wave Wc which is a repetitive wave having a cycle of a vibrating wave of a heartbeat as a basic cycle. The vibrating wave Wc represents the autocorrelation of the original vibrating wave W0, and the smaller the value of which, the higher the autocorrelation.

Since a difference between the original vibrating waves W0 is 0, a total S0 thereof is also 0. For example, if a waveform, which is deviated by one cycle of a heartbeat from the vibrating wave W0, is the vibrating wave Wi, the vibrating wave W0 and the vibrating wave Wi have the same or similar waveforms, thus a total Si of an absolute value of a difference will be 0 or a value close to 0. As shown in FIG. 5B, Si has a total next smaller than that of S0 and a period between S0 and Si corresponds to one cycle of a heartbeat.

The correlation computation unit 163 outputs the delayed vibrating waves Wi during the computation period Tc.

For example, when a delay time of the vibrating wave W0 is 1/32 seconds and the computation period Tc is 8 seconds, the correlation computation unit 163 outputs the vibrating waves W1 to W255. Since the sampling interval is 1/32 seconds which is the same as the delay time, 256 samplings are performed during the computation period Tc.

The correlation computation unit 163 computes a heart rate using a cycle of a peak at which a difference between a vibrating wave which has not been subjected to a delay and each vibrating wave which has been subjected to a delay is reduced in a waveform of the difference. To be specific, the correlation computation unit 163 determines a period from a time at which the waveform of the difference starts to a time of a first peak where the difference is reduced, as a cycle of a heartbeat. The correlation computation unit 163 computes and outputs a heart rate from the determined cycle of the heartbeat. Since multiple peaks at which the difference is reduced in the waveform of the difference appear, the correlation computation unit 163 may compute the heart rate in accordance with a period between the peaks. However, it is desirable to compute the heart rate from the first peak as described above because which increases the reliability of the heart rate.

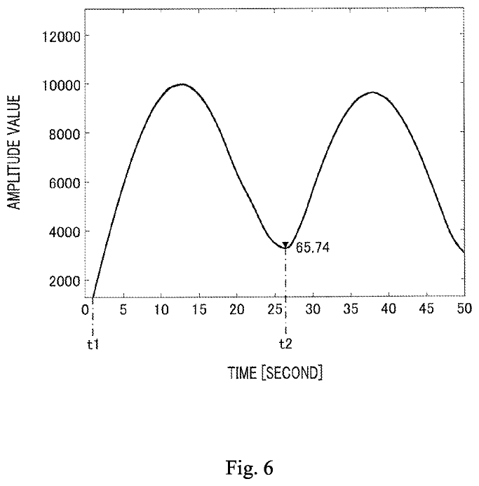

FIG. 6 illustrates a waveform of a difference between a vibrating wave which has not been subjected to a delay and each vibrating wave which has been subjected to a delay.

As shown in FIG. 6, a period from a time t1 at which the waveform of the difference starts to a time t2 of a first peak at which the difference is reduced is one cycle of a heartbeat. In the example of FIG. 6, the computation result in which a heart rate is 65.74 (BPM) is obtained from a time difference (t2−t1).

The determination unit 17 determines the reliability of the heart rate detected by the heartbeat detection unit 16. For example, the determination unit 17 calculates a variance value of the five most recent heart rates detected by the heartbeat detection unit 16. The determination unit 17 can determine the reliability to be high when the variance value is less than a threshold value, and can determine the reliability to be low when the variance value is the threshold value or more. The reliability may be divided into a plurality of levels. For example, the determination unit 17 can also determine the reliability in three levels using a plurality of threshold values for the variance value.

Also, the determination unit 17 can determine the reliability to be high when a heart rate is within a certain range, for example, 30 to 150 (BPM), and can determine the reliability to be low when the heart rate is outside of the certain range. The determination unit 17 can also compute or obtain an average heart rate of the user and determine the reliability depending on whether the detected heart rate is within a certain range from the average heart rate.

In the waveform of the difference between a vibrating wave which has not been subjected to a delay and each vibrating wave which has been subjected to a delay, when a peak apex value used for determining a cycle of a heartbeat is reduced, the waveform of the difference is closer to the vibrating wave of the heartbeat. Thus, the determination unit 17 can determine the reliability to be high when the peak apex value used for determining the cycle of the heartbeat is lower than a certain value, and can determine the reliability to be low when the peak apex value used for determining the cycle of the heartbeat is the certain value or more.

The determination unit 17 outputs the determined reliability together with the heart rate detected by the heartbeat detection unit 16. When the display device 3 displays a heart rate, it is possible to display the heart rate together with the reliability. The heart rate may be displayed in a display form according to the reliability. For example, when a heart rate is displayed, it is possible to display a heart rate with high reliability in black and display a heart rate with low reliability in red.

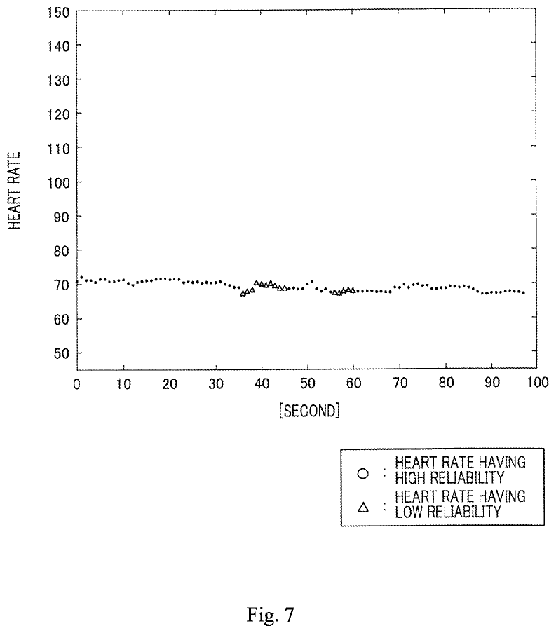

FIG. 7 illustrates a display example of a heart rate.

As shown in FIG. 7, a plot of a heart rate detected by the heartbeat detection device 1 at certain time intervals is displayed in chronological order. Among the heart rates, a heart rate determined to be high reliability is displayed with a circle marker and a heart rate determined to be low reliability is displayed with a triangular marker.

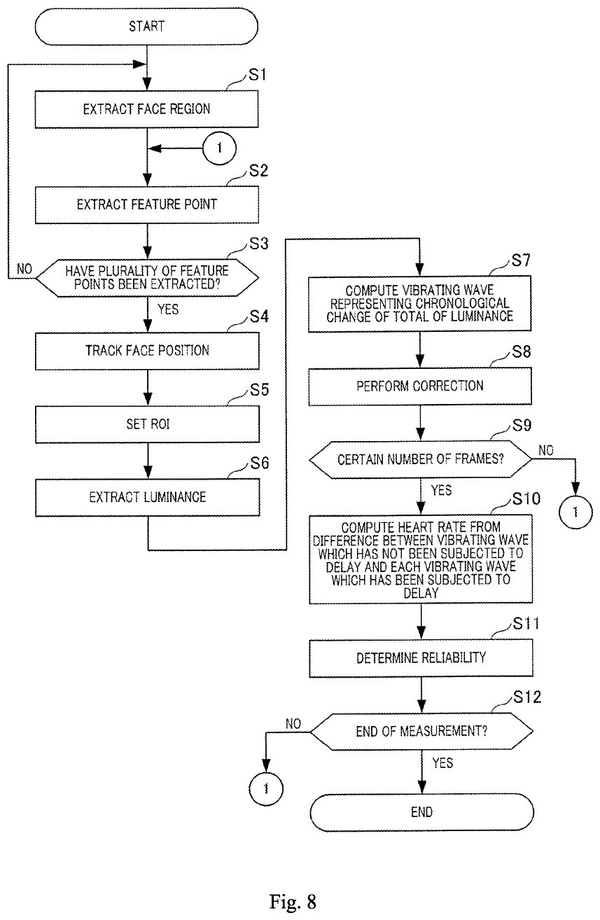

FIG. 8 is a flowchart for describing a processing procedure when a heartbeat is detected in the above-described heartbeat detection device 1.

In the heartbeat detection device 1, as shown in FIG. 8, the face extraction unit 11 extracts a face region from a captured image of a user's body surface input from the imaging device 2 (Step S1). The feature point extraction unit 12 extracts a feature point from the detected face region (Step S2). As a result, when a plurality of feature points are not extracted (Step S3: NO), the process returns to the process of Step S1.

When the plurality of feature points are extracted (Step S3: YES), the tracking unit 13 determines a degree of similarity between each feature point extracted in the captured image of a current frame and each feature point extracted in the captured image of an immediately previous frame. The tracking unit 13 performs projective transformation on the captured image of the current frame so that positions of the feature points having the highest degree of similarity match and causes the face position of the current frame to track the face position of the immediately previous frame (Step S4). Through the tracking, it is possible to reduce a noise component due to the movement of the user in the vibrating wave representing the chronological change of the luminance in the captured image.

The ROI setting unit 14 sets an ROI in the captured image of the current frame which has been subjected to the tracking of the face position (Step S5). On the other hand, the luminance extraction unit 15 extracts the luminance in G from the captured image input from the imaging device 2 (Step S6).

In the heartbeat detection unit 16, the integration computation unit 161 computes a total luminance in G in the set ROI and stores it in a memory. The integration computation unit 161 reads the total luminance in G within a certain period from the memory and computes the vibrating wave representing the chronological change of each read total luminance (Step S7). The correction unit 162 corrects this vibrating wave (Step S8). Here, when the number of frames of the captured image for which the vibrating wave is computed has not reached a certain number and the vibrating wave corresponding to the computation period Ts has not yet been obtained (Step S9: NO), the process returns to the process of Step S2.

On the other hand, when the number of frames of the captured image for which the vibrating wave is computed reaches the certain number and the vibrating wave corresponding to the computation period Ts is obtained (Step S9: YES), the correlation computation unit 163 delays the vibrating wave which has been subjected to the correction process at certain time intervals and obtains a waveform of the difference between the vibrating wave which has not been subjected to a delay and each vibrating wave which has been subjected to a delay. The correlation computation unit 163 computes a heart rate, as one cycle of a heartbeat, using a period from a time at which the waveform of the difference starts to a time at which a first peak where the difference is reduced appears in the waveform of the difference (Step S10).

The determination unit 17 determines the reliability of the heart rate computed using the heartbeat detection unit 16 (Step S11). The heart rate computed using the heartbeat detection unit 16 is output to the display device 3 together with the reliability determined by the determination unit 17. The output heart rate is displayed on the display device 3 in a display form such as a numerical value, a graph, or the like. The display form of the heart rate can be changed in accordance with the reliability.

When an instruction to end the measurement of the heart rate is not given (Step S12: NO), the process returns to the process of Step S2. When the instruction to end the measurement is given (Step S12: YES), this process ends.

As described above, the heartbeat detection device 1 in this embodiment includes the heartbeat detection unit 16 which detects a heart rate using the luminance of captured images of a part of the body surface of the user, which are captured images of the plurality of frames captured in chronological order. The heartbeat detection unit 16 computes the total of the luminance of the captured image of each of the frames, delays the vibrating wave representing the chronological change of the total of the luminance at certain time intervals, and computes the heart rate using the cycle of the peak at which the difference between the vibrating wave which has not been subjected to a delay and each vibrating wave which has been subjected to a delay is reduced in the waveform of the difference.

According to the above-described embodiment, the vibrating wave component having the periodicity of the heartbeat is obtained from the difference between the vibrating wave which has not been subjected to a delay and each vibrating wave which has been subjected to a delay. Thus, even when a vibrating wave component of a long cycle due to the movement of the user is included in each of the vibrating waves, it is possible to detect a heart rate with high accuracy. Furthermore, since the heart rate can be computed through the simple computation of the addition of the luminance and the subtraction of each vibrating wave, it is possible to detect the heart rate with a small amount of computation. Therefore, it is also possible to shorten a time for detection of a heart rate.

When the heart rate is computed by performing frequency conversion such as Fourier transformation or wavelet transformation on the vibrating wave representing the chronological change of the luminance, it is difficult to obtain a cycle of a heartbeat with the number of samplings of about 256 points as in this embodiment. A larger number of samplings is required to obtain sufficient heart rate detection accuracy. Furthermore, the frequency conversion is more easily affected by the vibrating wave component having a longer cycle than that of the heartbeat and reduces the resolution. Thus, it is difficult to extract the vibrating wave component of the heartbeat with high accuracy.

Even when the heart rate is computed using an autocorrelation function for the vibrating wave representing the chronological change of the luminance, it is difficult to extract the vibrating wave of the heartbeat with high accuracy, due to the influence of the vibrating wave component having a longer cycle than that of the heartbeat. The autocorrelation function is generally expressed by an expression, i.e., R(t,s)=E[(Xt−μ)(Xs−μ)]/σ² (Xt and Xs represent values at times t and s, μ represents an average of Xt, σ² represents a variance, and E represents an expected value).

On the other hand, according to this embodiment, since the cycle of the heartbeat is obtained from the difference of each delayed vibrating wave, the influence of the vibrating wave component of a long cycle is reduced and it is possible to compute the cycle of the heartbeat with high accuracy. Furthermore, according to this embodiment, since it is possible to detect a heart rate only by addition and subtraction and an amount of computation is small as compared with frequency conversion, an autocorrelation function, and the like in which complex computation using multiplication, division, or functions is required, it is possible to shorten a time for detection.

The above-described embodiment is a preferred example of the present invention and is not limited thereto. It is possible to appropriately perform change within the scope of the technical idea of the present invention.

For example, the captured image which can be used for detecting the heart rate is not limited to the captured image having the luminance in R, G, and B described above and may be a captured image having the luminance of a color space other than R, G, and B such as L*, a*, and b*. Furthermore, the luminance extraction unit 15 may extract the luminance obtained by weighting and averaging the luminance in R, G, and B, the luminance representing the brightness, or the like, as the luminance to be used for detecting a heart rate. According to the present invention, it is possible to detect a heart rate with high accuracy even if the luminance is other than the luminance in G.

Also, if a captured image to be used for detecting a heart rate is a captured image of a part of a body surface of a user, for example, the captured image may be a captured image of a body surface of a part other than the face such as the wrist, the back of the hand, or the neck, instead of a captured image of the face.

Priority is claimed on Japanese Patent Application No. 2018-122754, filed on Jun. 28, 2018, and all the contents of which are incorporated herein by reference.

### REFERENCE SIGNS LIST

1 Heartbeat detection device

11 Face extraction unit

12 Feature point extraction unit

13 Tracking unit

14 ROI setting unit

16 Heartbeat detection unit

161 Integration computation unit

162 Correction unit

163 Correlation computation unit

17 Determination unit

1. A heartbeat detection device, comprising:
a heartbeat detector which detects a heart rate using a luminance of captured images of a part of a body surface of a user, which are captured images of a plurality of frames which have been captured in chronological order, wherein
the heartbeat detector computes a total luminance of the captured image of each of the frames, delays a vibrating wave representing chronological change of the total luminance at certain time intervals, and computes the heart rate using a cycle of a peak at which a difference between the vibrating wave which has not been subjected to a delay and each vibrating wave which has been subjected to a delay is reduced in a waveform of the difference.

2. The heartbeat detection device according to claim 1, wherein
the heartbeat detector computes the heart rate using a period from a time at which the waveform of the difference starts to a time at which the peak appears first, as one cycle.

3. The heartbeat detection device according to claim 1, further comprising:
a determiner which determines a reliability of the heart rate detected by the heartbeat detector and outputs the reliability together with the heart rate.

4. The heartbeat detection device according to claim 1, wherein
the luminance is a luminance in green.

5. The heartbeat detection device according to claim 1, further comprising:
a region of interest (ROI) setter which sets an ROI in the captured image, wherein
the heartbeat detector computes a total luminance in the ROI.

6. The heartbeat detection device according to claim 1, wherein
the captured image is a captured image of a face of the user, and
the heartbeat detection device includes:
a feature point extractor which extracts a feature point of the face in the captured image of each of the frames; and
a tracker which uses the feature point to adjust a face position in the captured image of each of the frames.

7. A heartbeat detection method, comprising:
detecting a heart rate using a luminance of captured images of a part of a body surface of a user, which are captured images of a plurality of frames which have been captured in chronological order, wherein
the detecting the heart rate includes:
computing a total luminance of the captured image of each of the frames;
delaying a vibrating wave representing chronological change of the total luminance at certain time intervals; and
computing the heart rate using a cycle of a peak at which a difference between the vibrating wave which has not been subjected to a delay and each vibrating wave which has been subjected to a delay is reduced in a waveform of the difference.

8. A non-transitory computer-readable medium storing a program for causing a computer to execute:
detecting a heart rate using a luminance of captured images of a part of a body surface of a user, which are captured images of a plurality of frames which have been captured in chronological order, wherein
the detecting the heart rate includes:
computing a total luminance of the captured image of each of the frames;
delaying a vibrating wave representing chronological change of the total luminance at certain time intervals; and
computing the heart rate using a cycle of a peak at which a difference between the vibrating wave which has not been subjected to a delay and each vibrating wave which has been subjected to a delay is reduced in a waveform of the difference.
